(19) United States
(12) Patent Application Publication
(10) Pub. No.: US 2024/0424048 A1
(43) Pub. Date: Dec. 26, 2024

(54) CHINESE MEDICINE FORMULA FOR TREATING TUMORS AND/OR PULMONARY NODULES AND PREPARATION METHOD THEREOF

(71) Applicants: SHUNJU BI, Nanhu New Village (CN); XIANGQUN CHEN, Changchun City (CN)

(72) Inventors: SHUNJU BI, Nanhu New Village (CN); XIANGQUN CHEN, Changchun City (CN)

(21) Appl. No.: 18/746,293

(22) Filed: Jun. 18, 2024

(30) Foreign Application Priority Data
Jun. 20, 2023 (CN) 202310732035.3

(51) Int. Cl. A61K 36/704; A61K 35/644; A61K 36/074; A61K 36/185; A61K 36/254; A61K 36/708; A61K 36/73; A61K 36/87; A61K 36/8965; A61K 36/898; A61P 35/00

(52) U.S. Cl. CPC A61K 36/704; A61K 35/644; A61K 36/074; A61K 36/185; A61K 36/254; A61K 36/708; A61K 36/73; A61K 36/87; A61K 36/8965; A61K 36/898; A61P 35/00

(57) ABSTRACT

A Chinese medicinal formula for treating tumors and/or pulmonary nodules and a preparation method thereof are provided. The Chinese medicinal formula is prepared from the following raw materials: Acanthopanax gracilistylus 20-30 g, Polygonum multiflorum 15-25 g, glossy ganoderma 15-25 g, snow pear 15-25 g, Gymnadenia conopsea 15-25 g, honey 450-550 g, Rheum officinale 15-25 g, Rhizoma polygonati 15-25 g, hanconggou 15-25 g and white wine 800-1200 ml; the Chinese medicine formula of the present disclosure can significantly improve the human immune system, has no toxic side effects, and has the effects of inhibiting tumors and pulmonary nodules, and anti-fatigue. It has a dual effect, high efficiency, good effect, and low cost. It can be applied to a clinical treatment of mid to late stage lung cancer, especially for subjects who are not suitable or have not yet undergone surgical resection, and is a good for the treatment of lung cancer.

### CROSS-REFERENCE TO RELATED APPLICATIONS

This application claims priority to Chinese Patent Application No. 202310732035.3, filed on Jun. 20, 2023, which is hereby incorporated by reference in its entirety.

### TECHNICAL FIELD

The present disclosure relates to the field of Chinese medicine technologies, and in particular, to a Chinese medicine formula for treating tumors and/or pulmonary nodules and a preparation method thereof.

### BACKGROUND

Modern industrial civilization has emerged a large amount of carcinogens, with over 60000 toxic substances worldwide and over 2000 new synthesized toxic substances; artificial pesticides such as aluminum, mercury, cadmium, zinc, nickel, arsenic, sulfuric acid aerosols, benzene hexachloride, dieldrin, and DDT (dichloro-diphenyl-trichloroethane) enter the human body through the atmosphere, water, soil, food, etc., which causes a continuous increase in cancer mortality rates, rendering it the largest disease that endangers human life.

The current treatment for lung cancer mainly adopts traditional Western medicine surgical resection, as well as postoperative chemotherapy and radiation therapy, which have advantages and disadvantages. It does have therapeutic effects on some early detected cancers; however, for mid-stage, late-stage, or elderly and frail patients, the postoperative chemotherapy and radiation therapy not only kill cancer cells but also beneficial cells, and develop drug resistance, exacerbate the already low immunity of patients. Although the tumor has been removed, the generation source is still present, which just cures the symptoms. After discontinuing medication, it is prone to recurrence and metastasis, and lead to low survival rates for patients.

Under current environmental pollution conditions, human pulmonary nodule symptoms are highly prevalent, especially an occurrence of malignant transformation of ground glass nodules in the lungs. However, Western medicine does not have a specific drug for treating pulmonary nodules in clinical practice and can only surgically remove the tissue of the pulmonary nodules. However, the postoperative recurrence rate is very high, with data indicating a high rate of 84%.

In view of this, the present disclosure is proposed.

### SUMMARY

The purpose of the present disclosure is to provide a Chinese medicine formula for treating tumors and/or pulmonary nodules and a preparation method thereof. The Chinese medicine formula adheres to the essence of “supplementing first and then attacking, supporting the right and eliminating evil” in traditional Chinese medicine, and is based on the ancestral secret formula combined with improvement, in order to comprehensively enhance the patient's human body function, improve their immunity and the activity of human macrophages, relieve wheezing by tonifying the lung, reduce swelling and relieve pain. In the treatment, the function for lung tumors and pulmonary nodules can make up for the shortcomings of Western medicine, achieve complementary advantages, and achieve positive and significant effects.

In order to achieve the above objectives of the present disclosure, the following technical solutions are adopted.

A first aspect of the present disclosure provides a Chinese medicine formula for treating tumors and/or pulmonary nodules, which is prepared from the following raw materials:
Acanthopanax gracilistylus 20-30 g, Polygonum multiflorum 15-25 g, glossy ganoderma 15-25 g, snow pear 15-25 g, Gymnadenia conopsea 15-25 g, honey 450-550 g, Rheum officinale 15-25 g, Rhizoma polygonati 15-25 g, hanconggou 15-25 g and white wine 800-1200 ml.

The pharmacology of the Chinese medicine formula of the present disclosure is as follows:
glossy ganoderma has sweet and bitter taste, mild nature, and belongs to the meridians of heart, lungs, liver, and spleen; it can nourish heart and calm mind, quiet lungs and replenish ki, regulate ki and eliminate stasis, nourish liver and strengthen spleen. Glossy ganoderma contains germanium, which can increase the ability of the human blood to absorb oxygen by 1.5 times and promote metabolism; organic germanium in the glossy ganoderma can induce production and activation of NK cells and macrophage activity in the human body, which participates in immune regulation; polysaccharides in the glossy ganoderma have bidirectional regulatory effects on the body's immune system, anti-tumor and protecting liver, rendering it a good medicine for treating tumors.

Acanthopanax gracilistylus (wilsonii) can moisten the lungs and strengthen the body, and can increase the body's oxygen absorption (its antioxidant capacity is five times that of vitamin E); wilsonii can resist fatigue and radiation, tonify weakness, enhance bone marrow hematopoietic function, and has an elevated effect on leukocyte caused by chemotherapy and radiation therapy.

Polygonum multiflorum has functions of detoxification, reducing carbuncle, moistening intestines, and promoting bowel movements; is good for tuberculosis, carbuncle and toxic sores, and hematochezia.

Gymnadenia conopsea, also known as Gymnadenia conopsea R. Brown, has a sweet taste; mild nature, belongs to the meridians of lungs, spleen, and stomach, with the function of tonify ki and replenish blood, used for lung deficiency cough and asthma, consumptive disease and emaciation, and neurasthenia.

Snow pear contains a variety of vitamins, which can moisten the lungs and clear dryness, stop coughing and dissolve phlegm, nourish blood and muscle; selenium element in the snow pear can play a role in cancer prevention and anticancer; polysaccharides in the snow pear can promote appetite, accelerate intestinal peristalsis and enhance intestinal function.

Mountain honey contains various inorganic salts and organic acids such as vitamins, iron, calcium, copper, manganese, potassium, phosphorus, and trace elements that are similar in concentration to human serum, as well as beneficial to human health. It can resist bacteria and inflammation, promote tissue regeneration, and enhance patient physical fitness. The multiple enzymes and minerals contained in the honey can synergistically enhance the human immunity.

Rheum officinale has a cold nature and has effects of clearing heat, laxative and eliminate accumulated stagnation in the body. It can be used to treat symptoms such as dry stools, difficulty in defecation, bloating, and abdominal pain caused by heat accumulation in the gastrointestinal tract; the Rheum officinale has a bitter taste and has effect of cooling blood and stopping bleeding, which helps to alleviate symptoms such as hematemesis, epistaxis, hemoptysis, sore throat, redness swelling, and pain of the eye caused by blood heat. Anthraquinone compounds contained in Rheum officinale can promote platelet formation, shorten coagulation time, and facilitate hemostasis.

Rhizoma polygonati has effects of tonifying the spleen and moistening the lungs, strengthening the spleen and kidneys, nourishing ki, etc. It can improve the body's immunity, enhance disease resistance, regulate blood sugar, eliminate free radicals, delay aging, and also alleviate fatigue, inhibit bacteria, dilate blood vessels, and promote blood circulation. The Rhizoma polygonati can be used to moisturize the lungs and relieve cough, and it is a warm medicinal herb that does not cause irritation to the body. If poor dietary habits in daily life led to symptoms such as lung heat, cough, and phlegm, the use of Rhizoma polygonati can have a regulating effect.

Hanconggou, as recorded in early ancient books, is a plant with superior effects than ginseng. It not only has effects of clearing the lungs and brightening the eyes, but also has hemostatic effects. Overall, hanconggou has many effects and purposes. Taking hanconggou regularly can dispel cold and dampness, strengthen the body, and prolong life.

Pure grain white wine has bitter, sweet, hot taste. Modern pharmacological studies of Chinese medicine show that white wine is a good organic solvent, and various components of drugs, such as salts, glycosides, tannins, physalin, organic acids, volatile liquor, resins, and sugars, are easy to dissolve in wine. Therefore, after the drug is processed by white wine, it helps to dissolve effective ingredients, promote blood circulation, and enhance the curative effect.

The Chinese medicine formula of the present disclosure adheres to the principle of “supplementing first, attacking later, supporting the right and eliminating evil” in Chinese medicine. It is based on the ancestral secret formula and improved by fusion, and high-quality medicinal materials are collected from the forest areas of Changbai Mountain and Daxing'an Mountains. The formulated formula comprehensively enhances the patient's human body function, improves immunity and the activity of human macrophages. It is warm and tonifying ki, tonifying the lungs and calming asthma, tonifying the kidneys, dispersing nodules and reducing swelling, and purifying blood. In the treatment of lung tumors and nodules, it can make up for the shortcomings of Western medicine, achieve complementary advantages, and achieve positive and significant effects.

In an embodiment of the present disclosure, the traditional Chinese medicine formula is prepared from the following raw materials:
Acanthopanax gracilistylus 25 g, Polygonum multiflorum 20 g, glossy ganoderma 20 g, snow pear 20 g, Gymnadenia conopsea 15-25 g, honey 500 g, Rheum officinale 20 g, Rhizoma polygonati 20 g, hanconggou 20 g and white wine 1000 ml.

In an embodiment of the present disclosure, the white wine is pure grain white wine and has an alcohol degree of 30°-53°.

In an embodiment of the present disclosure, the honey is mountain honey.

In an embodiment of the present disclosure, the tumors include lung cancer, liver cancer, kidney cancer, melanoma, gastric cancer, esophageal cancer, and lymphatic cancer.

A second aspect of the present disclosure provides a preparation method for the Chinese medicine formula, which includes the following steps:
(a) weighing each raw material;
(b) placing Acanthopanax gracilistylus, Polygonum multiflorum, glossy ganoderma, snow pear, Gymnadenia conopsea, honey, Rheum officinale, Rhizoma polygonati, and hanconggou into white wine, conducting soaking to obtain the Chinese medicinal formula.

In an embodiment of the present disclosure, a temperature for soaking is 23-25° C. and a time for soaking is 30-40 days.

In an embodiment of the present disclosure, the white wine is pure grain white wine and has an alcohol degree of 30°-53°.

The preparation method of the Chinese medicine formula of the present disclosure involves processing medicinal materials at a temperature of 23-25° C. for 30-40 days, which is similar to biological fermentation technology and can improve the medicinal properties.

Compared with prior art, the beneficial effects of the present disclosure at least include:
the Chinese medicine formula of the present disclosure has a novel and unique medicinal composition, which can significantly improve the human immune system, has no toxic side effects, and has the effects of first supplementing and then attacking, inhibiting tumors and pulmonary nodules, and anti-fatigue. It has a dual effect, high efficiency, good effect, and low cost. It can be applied to a clinical treatment of middle and late stage lung cancer, especially for subjects who are not suitable or have not yet undergone surgical resection, and is a good for the treatment of lung cancer; besides, it also has a significant therapeutic effect on pulmonary nodules; has a significant effect on the treatment of rectal cancer and lymphatic cancer, and can be applied to the treatment of liver cancer, kidney cancer, melanoma, esophageal cancer, and gastric cancer, and can produce positive effects; it can be applied to the treatment of pulmonary nodules with good results, or can be applied to the treatment of thyroid nodules with good results.

### BRIEF DESCRIPTION OF DRAWINGS

In order to provide a clearer explanation of specific embodiments of the present disclosure or the technical solutions in the prior art, the following will briefly introduce the accompanying drawings required in the specific embodiments or prior art descriptions. In all drawings, similar components or parts are generally identified by similar reference number. In the attached drawings, each component or part may not necessarily be drawn to an actual scale.

FIG. 1 shows a volume growth curve of Lewis lung cancer tumors in a first batch of experiments of the present disclosure.

FIG. 2 shows the volume growth curve of Lewis lung cancer tumors in a second batch of the experiments of the present disclosure.

### DESCRIPTION OF EMBODIMENTS

The following will provide a detailed description of the embodiments of the technical solution of the present disclosure in combination with the embodiments. The following embodiments are only intended to provide a clearer explanation of the technical solution of the present disclosure, and therefore are only used as examples and cannot be used to limit the protection scope of the present disclosure.

It should be noted that, unless otherwise specified, the technical or scientific terms used in the present application shall have the usual meaning understood by those skilled in the art to which the present disclosure belongs.

The raw materials used in the following examples, unless otherwise specified, can be purchased from the market;
mountain honey: Changbai Mountain wild honey, commercially available for purchase;
white wine: white wine is brewed from sorghum and purchased on the market.
white wine with other alcohol degree can be obtained by blending 53° white wine with distilled water.

### Example 1

This Example is a Chinese medicine formula for treating tumors and/or pulmonary nodules, and it is prepared from the following raw materials:
Acanthopanax gracilistylus 20 g, Polygonum multiflorum 25 g, glossy ganoderma 15 g, snow pear 25 g, Gymnadenia conopsea 15 g, honey 550 g, Rheum officinale 15 g, Rhizoma polygonati 25 g, hanconggou 15 g and white wine 1200 ml;
the white wine has an alcohol degree of 53°;
a preparation method of the Chinese medicine formula includes the following steps:
(a) weighing each raw material;
(b) placing Acanthopanax gracilistylus, Polygonum multiflorum, glossy ganoderma, snow pear, Gymnadenia conopsea, honey, Rheum officinale, Rhizoma polygonati, and hanconggou into white wine and conducting soaking at 23-25° C. for 30 days to obtain the Chinese medicinal formula.

### Example 2

This Example is a Chinese medicine formula for treating tumors and/or pulmonary nodules, it is prepared from the following raw materials:
Acanthopanax gracilistylus 30 g, Polygonum multiflorum 15 g, glossy ganoderma 25 g, snow pear 15 g, Gymnadenia conopsea 25 g, honey 450 g, Rheum officinale 25 g, Rhizoma polygonati 15 g, hanconggou 25 g and white wine 800 ml;
the white wine has an alcohol degree of 30°;
a preparation method of the Chinese medicine formula includes the following steps:
(a) weighing each raw material;
(b) placing Acanthopanax gracilistylus, Polygonum multiflorum, glossy ganoderma, snow pear, Gymnadenia conopsea, honey, Rheum officinale, Rhizoma polygonati, and hanconggou into white wine and conducting soaking at 23-25° C. for 40 days to obtain the Chinese medicinal formula.

### Example 3

This Example is a Chinese medicine formula for treating tumors and/or pulmonary nodules, it is prepared from the following raw materials:
Acanthopanax gracilistylus 25 g, Polygonum multiflorum 20 g, glossy ganoderma 20 g, snow pear 20 g, Gymnadenia conopsea 15-25 g, honey 500 g, Rheum officinale 20 g, Rhizoma polygonati 20 g, hanconggou 20 g and white wine 1000 ml;
the white wine has an alcohol degree of 35°;
a preparation method of the Chinese medicine formula includes the following steps:
(a) weighing each raw material;
(b) placing Acanthopanax gracilistylus, Polygonum multiflorum, glossy ganoderma, snow pear, Gymnadenia conopsea, honey, Rheum officinale, Rhizoma polygonati, and hanconggou into white wine and conducting soaking at 23-25° C. for 35 days to obtain the Chinese medicinal formula.

### Experimental Example

This experimental example is an efficacy test of the Chinese medicine formula prepared in Example 3 of the present disclosure on lung cancer:

### 1 Experimental Material

### 1.1 Medications

white wine (alcohol concentration: 53 degrees), the alcohol concentration was adjusted to 35 degrees with distilled water; an anti-lung cancer vinum (alcohol concentration: 35 degrees) was prepared by the preparation method in Example 3. When used, 10%, 20%, 40% and 80% of anti-lung cancer vinum were prepared with white wine having 35 degrees. Cyclophosphamide for injection (specification: 0.2 g/bottle) provided by Baxter Oncology GmbH, batch number OL435A, it was diluted to a solution (mass fraction of 0.075%) with sodium chloride injection (mass of 0.9% fraction) during use.

### 1.2 Animals

C57BL/6 mice, 6-8 weeks old, weighing 19-21 g, male, provided by Liaoning Changsheng Biotechnology Co., Ltd., certificate number: SYSK (Ji) 2018-0001. Two batches of experiments all used male C57BL/6 mice, which were adaptively raised in a laboratory for 3-5 days before the experiment.

### 1.3 Cell Lines and Cell Culture

Mouse Lewis lung cancer cells were provided by Jilin Cancer Research Institute and passaged and preserved in the laboratory. Lewis lung cancer cells were resuscitated, cells were expanded to 2-3×10⁸ cells/mL, cells were collected, and cell density was adjusted to 2×10⁷ cells/mL.

### 1.4 Animal Grouping and Administration Plan

a first batch of experiments: C57BL/6 mice were randomly divided into 6 groups according to body weight.
Saline control group administered with physiological saline 20 ml/kg
Solvent control group administered with 35-degree white wine 20 ml/kg
Cyclophosphamide administered with 0.075% 15 mg/20 group cyclophosphamide ml/kg
Low dose group of anti-administered with 20% anti-lung cancer 4 ml/20 lung cancer vinum ml/kg
Mid dose group of anti-administered with 40% anti-lung cancer 8 ml/20 lung cancer vinum ml/kg
High dose group of anti-Administered with 80% anti-lung 16 ml/20 m lung cancer vinum cancer vinum l/kg
a second batch of experiments: randomly divided into 6 groups based on body weight:
Saline control group administered with physiological saline 20 ml/kg
Solvent control group administered with 35-degree white wine 20 ml/kg
Cyclophosphamide administered with 0.075% 15 mg/20 group cyclophosphamide ml/kg
Low dose group of anti-administered with 10% anti-lung cancer 2 ml/20 lung cancer vinum ml/kg
Mid dose group of anti-administered with 20% anti-lung cancer 4 ml/20 lung cancer vinum ml/kg
High dose group of anti-administered with 40% anti-lung cancer 8 ml/20 lung cancer vinum ml/kg

### 1.5 Experimental Methods

C57BL/6 mice were adaptively fed for 3-5 days, and an in vivo transplant tumor model was prepared using anti-tumor drug experimental methods. Each mouse was subcutaneously injected with 0.2 ml of cell suspension with a concentration of 2×10⁷ cells/mL. On the second day, they were randomly divided into 6 groups based on body weight, with 20 mice in each group. Each group started administration the next day after tumor inoculation, and the drugs in each group were freshly prepared and administered at a dose of 10 ml/kg, twice a day for 14, 16, or 21 consecutive days.

The first batch of experiments: measure a tumor volume and plot a tumor growth curve once on the 8th, 10th, 12th, and 14th day of administration; after 14 days of administration, 10 mice in each group were weighed and euthanized. Tumors, spleens, lungs, and thymus were removed, weighed, tumor inhibition rate and organ coefficient were calculated; after 21 days of administration, 6 mice in each group were perfused with physiological saline and their lungs were removed, fixed with Bouin solution and the number of metastatic lung nodules was examined under a microscope.

The second batch of experiments: measure the tumor volume and plot the tumor growth curve once on the 8th, 10th, 12th, 14th, and 16th day of administration; after 16 days of administration, 12 mice in each group were weighed and euthanized. Tumors, spleens, lungs, and thymus were removed, weighed, and the tumor inhibition rate and organ coefficient were calculated; after 21 days of administration, 6 mice in each group were perfused with physiological saline and their lungs were removed, fixed with Bouis solution and the number of metastatic lung nodules was examined under the microscope.

$$Tumor volume = 1 / 2 \times a \times {b}^{2} ( a , b are the long axis ( mm ) and \\ short axis ( mm ) of the tumor , respectively ) \\ Tumor suppression rate = ( 1 - average tumor \\ weight in experimental group / average tumor \\ weight in control group ) \times 100 \% \\ Organ coefficient = organ weight ( mg ) / body weight ( g ) \times 100.$$

### 2 Experimental Results

### 2.1 Results of the First Batch of Experimental

### 2.1.1 Effect of Anti-Lung Cancer Vinum on Tumor Volume of Lewis Lung Cancer in Mice

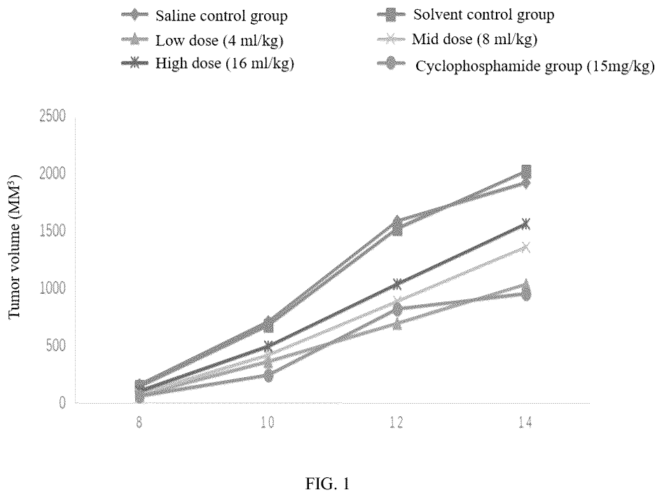

Compared with the saline control group, the solvent control group had no significant effect on the tumor volume of Lewis lung cancer mice after 8, 10, 12, and 14 days of administration (P>0.05). The cyclophosphamide group and the anti-lung cancer vinum group at a dose of 4 ml/kg could significantly reduce the tumor volume of Lewis lung cancer mice after 8, 10, 12, and 14 days of administration (P<0.05 or P<0.01). The anti-lung cancer vinum group at doses of 8, 16 ml/kg could significantly reduce the tumor volume of Lewis lung cancer mice after 8, 10, and 12 days of administration (P<0.05 or P<0.01); compared with the solvent control group, the cyclophosphamide group and the anti-lung cancer vinum group at doses of 4 and 8 ml/kg significantly reduced the tumor volume of Lewis lung cancer mice after 8, 10, 12, and 14 days of administration (P<0.05 or P<0.01). The anti-lung cancer vinum group at a dose of 16 ml/kg significantly reduced the tumor volume of Lewis lung cancer mice after 8, 10, and 12 days of administration (P<0.05), as shown in Table 1 and FIG. 1.

TABLE 1
Effect of anti-lung cancer vinum on the tumor volume of
Lewis lung cancer in mice (x̅ ± s)

| | Number | Volume of tumor (mm³) | | | |
|---|---|---|---|---|---|
| Groups | of animal (n) | 8 days | 10 days | 12 days | 14 days |
| Saline control | 16 | 157.41 | 714.28 | 1586.56 | 1921.20 |
| group 20 ml/kg | | | | | |
| Solvent control | 16 | 145.24 | 674.06 | 1515.20 | 2019.41 |
| group 20 ml/kg | | | | | |
| Cyclophosphamide | 16 | 61.08##** | 244.09##** | 817.83##** | 953.67##** |
| group 15 mg/kg | | | | | |
| Anti-lung cancer | | | | | |
| vinum 4 ml/kg | 16 | 72.05##** | 364.10##** | 969.21##** | 1033.74#** |
| 8 ml/kg | 16 | 81.88##** | 418.38##** | 882.42##** | 1358.56* |
| 16 ml/kg | 16 | 99.02#** | 495.83#* | 1037.57#* | 1558.33 |

compared with the saline control group, #P < 0.05, ##P < 0.01; compared with the solvent control group, *P < 0.05, **P < 0.01.

### 2.1.2 Inhibition of Anti-Lung Cancer Vinum on the Growth of Lewis Lung Cancer in Mice

Compared with the saline control group, the solvent control group and the positive drug cyclophosphamide group showed a significant decrease in body weight (P<0.05 or P<0.01), while there was no significant change in body weight among the anti-lung cancer vinum groups at three doses (P>0.05); compared with the solvent control group, the body weight of mice of the anti-lung cancer vinum groups at doses of 4, 8 ml/kg increased significantly (P<0.05 or P<0.01), while there was no significant change in body weight in the solvent control group, the anti-lung cancer vinum group at a dose of 16 ml/kg, and the positive drug cyclophosphamide group (P>0.05); compared with the saline control group, the tumor inhibition rate of the solvent control group was 3.56%, and the tumor inhibition rates of the anti-lung cancer vinum groups at doses of 4, 8, and 16 ml/kg were 48.02%, 35.46%, and 24.29%, respectively. This indicates that the anti-lung cancer vinum has a significant growth inhibitory effect on Lewis lung cancer in mice, as shown in Table 2.

TABLE 2
Effect of anti-lung cancer vinum on the tumor volume of Lewis lung cancer in mice (x̅ + s)

| | | Weight | Weight | | |
|---|---|---|---|---|---|
| | | before | after | | Tumor |
| | Number | administration | administration | Weight of | inhibition |
| Groups | of animal (n) | (g) | (g) | tumor (g) | rate (%) |
| Saline control | 10 | 19.23 ± 1.51 | 22.40 ± 1.65 | 1.68 ± 0.33 | — |
| group 20 ml/kg | | | | | |
| Solvent control | 10 | 19.23 ± 1.45 | 20.90 ± 0.88# | 1.62 ± 0.27 | 3.56 |
| group 20 ml/kg | | | | | |
| Cyclophosphamide | 10 | 19.23 ± 1.55 | 20.30 ± 1.33## | 0.59 ± 0.26** | 63.52 |
| group 15 mg/kg | | | | | |
| Anti-lung cancer | | | | | |
| vinum 4 ml/kg | 10 | 19.21 ± 1.47 | 22.20 ± 0.79** | 0.84 ± 0.29** | 48.02 |
| 8 ml/kg | 10 | 19.22 ± 1.48 | 21.90 ± 1.10* | 1.05 ± 0.18** | 35.46 |
| 16 ml/kg | 10 | 19.23 ± 1.44 | 21.50 ± 1.18 | 1.22 ± 0.39* | 24.29 |

compared with the saline control group, #P < 0.05, ##P < 0.01; compared with the solvent control group, *P < 0.05, **P < 0.01.

### 2.1.3 Effect of Anti-Lung Cancer Vinum on the Organ Coefficient of Lewis Lung Cancer in Mice

Compared with the saline control group, the thymus coefficient and spleen coefficient of mice for the positive drug cyclophosphamide group were significantly reduced (P<0.01), while the lung coefficient was significantly increased (P<0.05). There were no significant changes in the thymus coefficient, spleen coefficient, and lung coefficient in the solvent control group and the anti-lung cancer vinum group at three doses (P>0.05); compared with the solvent control group, the thymus coefficient and spleen coefficient of mice for the positive drug cyclophosphamide group were significantly reduced (P<0.05 or P<0.01), while the lung coefficient was significantly increased (P<0.05). There was no significant change in the thymus coefficient, spleen coefficient, and lung coefficient of the solvent control group and the anti-lung cancer vinum group at three doses (P>0.05), as shown in Table 3.

TABLE 3
Effect of anti-lung cancer vinum on the tumor volume of Lewis lung cancer in mice (x̅ + s)

| | | Weight | | | |
|---|---|---|---|---|---|
| | | before | | | |
| | Number | administration | Thymic | Spleen | Lung |
| Groups | of animal (n) | (g) | coefficient | coefficient | coefficient |
| Saline control | 10 | 22.40 ± 1.65 | 1.92 ± 0.48 | 5.85 ± 1.98 | 6.48 ± 0.73 |
| group 20 ml/kg | | | | | |
| Solvent control | 10 | 20.90 ± 0.88 | 1.45 ± 0.34 | 5.35 ± 1.03 | 6.46 ± 0.48 |
| group 20 ml/kg | | | | | |
| Cyclophosphamide | 10 | 20.30 ± 1.33 | 1.11 ± 0.35##* | 3.40 ± 1.02##** | 7.26 ± 0.85#* |
| group 15 mg/kg | | | | | |
| Anti-lung cancer | 10 | 22.20 ± 0.79 | 1.51 ± 0.38# | 4.37 ± 1.39 | 6.37 ± 0.28 |
| vinum 4 ml/kg | | | | | |
| 8 ml/kg | 10 | 21.90 ± 1.10 | 1.50 ± 0.19# | 4.84 ± 1.76 | 6.32 ± 0.51 |
| 16 ml/kg | 10 | 21.50 ± 1.18 | 1.59 ± 0.33 | 4.46 ± 1.95 | 6.59 ± 0.61 |

Compared with the saline control group, #P < 0.05, ##P < 0.01; compared with the solvent control group, *P < 0.05, **P < 0.01.

### 2.1.4 Effect of Anti-Lung Cancer Vinum on Lung Metastasis of Lewis Lung Cancer in Mice

Compared with the saline control group, both the positive drug cyclophosphamide group and the anti-lung cancer vinum group at three doses significantly reduced the number of lung nodules in mice (P<0.01). The solvent control group showed a decreasing trend in the number of lung nodules in mice, but there was no significant difference (P>0.05); compared with the solvent control group, both the positive drug cyclophosphamide group and the anti-lung cancer vinum group at three doses significantly reduced the number of lung nodules in mice (P<0.01), as shown in Table 4.

TABLE 4
Effect of anti-lung cancer vinum on the tumor volume of
Lewis lung cancer in mice (x̅ ± s)

| | Number | Weight after | |
|---|---|---|---|
| | of animal | administration | Number of |
| Groups | (n) | (g) | pulmonary nodules |
| Saline control group | 6 | 23.38 ± 1.44 | 17.67 ± 3.50 |
| 20 ml/kg | | | |
| Solvent control group | 6 | 22.95 ± 1.48 | 14.14 ± 3.31 |
| 20 ml/kg | | | |
| Cyclophosphamide | 6 | 21.81 ± 1.37 | 6.17 ± 1.94##** |
| group 15 mg/kg | | | |
| Anti-lung | | | |
| cancer vinum | | | |
| 4 ml/kg | 6 | 23.26 ± 1.83 | 7.17 ± 2.04##** |
| 8 ml/kg | 6 | 23.20 ± 2.14 | 7.83 ± 1.33##** |
| 16 ml/kg | 6 | 22.13 ± 2.08 | 8.17 ± 0.98##** |

compared with the saline control group, P < 0.01; compared with the solvent control group,
**P < 0.01.

### 2.2 Results of the Second Batch of Experiments

### 2.2.1 Effect of Anti-Lung Cancer Vinum on Tumor Volume of Lewis Lung Cancer in Mice

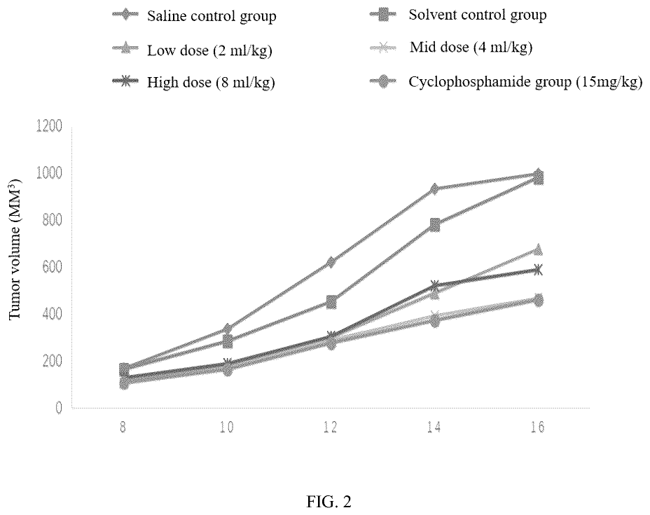

Compared with the saline control group, the solvent control group had no significant effect on the tumor volume of Lewis lung cancer mice after 8, 10, 12, 14, and 16 days of administration (P>0.05). The cyclophosphamide group and the anti-lung cancer vinum group at doses of 4 and 8 ml/kg could significantly reduce the tumor volume of Lewis lung cancer mice after 8, 10, 12, 14, and 16 days of administration (P<0.05 or P<0.01). The anti-lung cancer vinum group at a dose of 2 ml/kg could significantly reduce the tumor volume of Lewis lung cancer mice after 8, 10, 12, and 14 days of administration (P<0.05 or P<0.01). Compared with the solvent control group, the cyclophosphamide group and the anti-lung cancer vinum group at doses of 4 and 8 ml/kg significantly reduced the tumor volume of Lewis lung cancer mice after 8, 10, 12, 14, and 16 days of administration (P<0.05 or P<0.01). The anti-lung cancer vinum group at a dose of 2 ml/kg significantly reduced the tumor volume of Lewis lung cancer mice after 8, 10, 12, and 14 days of administration (P<0.05), as shown in Table 5 and FIG. 2.

TABLE 5
Effect of anti-lung cancer vinum on the tumor volume of Lewis lung cancer in mice (x̅ + s)

| | | Volume of tumor (mm³) | | | | |
|---|---|---|---|---|---|---|
| | Number | 8 | 10 | 12 | 14 | 16 |
| Groups | of animal (n) | days | days | days | days | days |
| Saline control | 18 | 170.39 | 338.44 | 621.09 | 931.57 | 995.73 |
| group 20 ml/kg | | | | | | |
| Solvent control | 18 | 164.63 | 283.84 | 451.59 | 781.18 | 982.52 |
| group 20 ml/kg | | | | | | |
| Cyclophosphamide | 18 | 111.30##** | 165.52##** | 278.25##** | 371.57##** | 461.54##** |
| group 15 mg/kg | | | | | | |
| Anti-lung cancer | 18 | 114.07#* | 179.07##* | 302.49##* | 488.07#* | 678.60 |
| vinum 2 ml/kg | | | | | | |
| 4 ml/kg | 18 | 105.16##** | 169.18##* | 291.18##* | 392.46##** | 471.02##** |
| 8 ml/kg | 18 | 128.31##* | 191.09##* | 306.79##* | 520.95#* | 589.99#* |

compared with the saline control group, #P < 0.05, ##P < 0.01; compared with the solvent control group, *P < 0.05, **P < 0.01.

2.2.2 Inhibition of Anti-Lung Cancer Vinum on the Growth of Lewis Lung Cancer in Mice

Compared with the saline control group, the body weight of mice in the solvent control group and the positive drug cyclophosphamide group decreased significantly (P<0.05 or P<0.01), while there was no significant change in body weight of mice among the anti-lung cancer vinum groups at dosage of 2, 4, and 8 ml/kg (P>0.05); compared with the saline control group, the tumor inhibition rate of the solvent control group was 8.20%, and the tumor inhibition rates of the anti-lung cancer vinum groups at doses of 2, 4, and 8 ml/kg were 23.98%, 49.02%, and 38.52%, respectively. This indicates that the anti-lung cancer vinum has a significant growth inhibitory effect on Lewis lung cancer in mice, as shown in Table 6.

TABLE 6
Effect of anti-lung cancer vinum on the tumor volume of Lewis lung cancer in mice (x̅ + s)

| | | Weight | Weight | | |
|---|---|---|---|---|---|
| | | before | after | | Tumor |
| | Number | administration | administration | Weight | inhibition rate |
| Groups | of animal (n) | (g) | (g) | of tumor (g) | (%) |
| Saline control | 12 | 19.32 ± 1.42 | 23.25 ± 0.97 | 1.88 ± 0.73 | — |
| group 20 ml/kg | | | | | |
| Solvent control | 12 | 19.37 ± 1.16 | 22.42 ± 0.79# | 1.72 ± 0.32 | 8.20 |
| group 20 ml/kg | | | | | |
| Cyclophosphamide | 12 | 19.33 ± 1.13 | 21.58 ± 0.79##* | 0.56 ± 0.22** | 67.43 |
| group 15 mg/kg | | | | | |
| Anti-lung cancer | | | | | |
| vinum 2 ml/kg | 12 | 19.30 ± 1.07 | 22.58 ± 1.08 | 1.31 ± 0.38* | 23.98 |
| 4 ml/kg | 12 | 19.38 ± 1.03 | 23.08 ± 0.67 | 0.88 ± 0.29** | 49.02 |
| 8 ml/kg | 12 | 19.35 ± 1.05 | 22.67 ± 0.78 | 1.06 ± 0.26** | 38.52 |

Compared with the saline control group, #P < 0.05, ##P < 0.01; compared with the solvent control group, **P < 0.05, **P < 0.01.

### 2.2.3 Effect of Anti-Lung Cancer Vinum on the Organ Coefficient of Lewis Lung Cancer in Mice

Compared with the saline control group, the thymus coefficient and spleen coefficient of mice for the positive drug cyclophosphamide group were significantly reduced (P<0.01), while the lung coefficient showed no significant change (P>0.05). The thymus coefficient, spleen coefficient, and lung coefficient of the solvent control group and the anti-lung cancer vinum groups at three doses did not show significant changes (P>0.05); compared with the solvent control group, the thymus coefficient and spleen coefficient of mice for the positive drug cyclophosphamide group were significantly reduced (P<0.05 or P<0.01), with no significant changes (P>0.05). The thymus coefficient, spleen coefficient, and lung coefficient of the solvent control group and the anti-lung cancer vinum group at three doses did not show significant changes (P>0.05), as shown in Table 7.

TABLE 7
Effect of anti-lung cancer vinum on the tumor volume of Lewis lung cancer in mice (x̅ + s)

| | | Weight after | | | |
|---|---|---|---|---|---|
| | Number | administration | Thymic | Spleen | Lung |
| Groups | of animal (n) | (g) | coefficient | coefficient | coefficient |
| Saline control | 10 | 23.25 ± 0.97 | 1.17 ± 0.23 | 6.25 ± 1.95 | 6.51 ± 0.56 |
| group 20 ml/kg | | | | | |
| Solvent control | 10 | 22.42 ± 0.79 | 1.03 ± 0.21 | 5.47 ± 1.70 | 6.19 ± 0.47 |
| group 20 ml/kg | | | | | |
| Cyclophosphamide | 10 | 21.58 ± 0.79 | 0.47 ± 0.16##** | 3.62 ± 0.86##* | 6.66 ± 0.72 |
| group 15 mg/kg | | | | | |
| Anti-lung cancer | 10 | 22.58 ± 1.08 | 1.06 ± 0.21 | 5.08 ± 1.92 | 6.41 ± 0.65 |
| vinum 2 ml/kg | | | | | |
| 4 ml/kg | 10 | 23.08 ± 0.67 | 1.02 ± 0.18 | 5.88 ± 1.71 | 6.22 ± 0.65 |
| 8 ml/kg | 10 | 22.67 ± 0.78 | 1.01 ± 0.28 | 5.56 ± 1.90 | 6.26 ± 0.32 |

compared with the saline control group, P < 0.01; compared with the solvent control group, **P < 0.01.

### 2.2.4 Effect of Anti-Lung Cancer Vinum on Lung Metastasis of Lewis Lung Cancer in Mice

Compared with the saline control group, both the positive drug cyclophosphamide group and the anti-lung cancer vinum group at three doses significantly reduced the number of lung nodules in mice (P<0.01). The solvent control group showed a decreasing trend in the number of lung nodules in mice, but there was no significant difference (P>0.05); compared with the solvent control group, both the positive drug cyclophosphamide group and the anti-lung cancer vinum group at three doses significantly reduced the number of lung nodules in mice (P<0.05 or P<0.01), as shown in Table 8.

TABLE 8
Effects of anti-lung cancer vinum on lung Metastasis
of Lewis Lung Cancer in Mice (±s)

| | Number | Weight after | |
|---|---|---|---|
| | of animal | administration | Number of |
| Groups | (n) | (g) | pulmonary nodules |
| Saline control group | 6 | 23.97 ± 1.32 | 14.33 ± 1.21 |
| 20 ml/kg | | | |
| Solvent control group | 6 | 23.13 ± 1.57 | 11.50 ± 2.88 |
| 20 ml/kg | | | |
| Cyclophosphamide | 6 | 22.02 ± 1.71 | 6.67 ± 1.21##** |
| group 15 mg/kg | | | |
| Anti-lung | | | |
| cancer vinum | | | |
| 2 ml/kg | 6 | 23.34 ± 1.55 | 7.33 ± 1.86##* |
| 4 ml/kg | 6 | 23.97 ± 1.85 | 5.17 ± 1.47##** |
| 8 ml/kg | 6 | 23.22 ± 1.64 | 6.83 ± 1.72##** |

Compared with the saline control group, P < 0.01; compared with the solvent control group,
**P < 0.01.

In summary, continuous gastric administration of 2, 4, 8, and 16 ml/kg anti-lung cancer vinum to mice for 8, 10, 12, and 14 days significantly reduced the tumor volume of Lewis lung cancer mice, and the tumor inhibition rates were 23.98%, 48.52%, 36.99%, and 24.29%, respectively. Continuous administration for 21 days significantly reduced the number of lung nodules in mice, which indicated that anti-lung cancer vinum can inhibit lung metastasis in Lewis lung cancer mice. According to the guidelines for the development of new Chinese medicine drugs, a tumor inhibition rate greater than 30% is considered effective. Anti-lung cancer vinum at doses of 4 and 8 ml/kg (these doses are equivalent to clinical doses of 30 and 60 ml/60 kg per person, respectively) have good anti-lung cancer activity. At this dose, there is no significant effect of mice body weight, thymus coefficient, spleen coefficient, and lung coefficient. The positive drug cyclophosphamide at a dose of 15 mg/kg was continuously administered by gavage to mice for 8, 10, 12, 14, and 16 days, which significantly reduced the tumor volume of Lewis lung cancer mice and had a tumor inhibition rate of 65.48%. Continuous administration for 21 days significantly reduced the number of lung nodules in mice, which indicates that it can inhibit lung metastasis in Lewis lung cancer mice. However, this dose can significantly reduce the body weight, thymic coefficient, and spleen coefficient of mice, which exhibits significant toxic effects.

Finally, it should be noted that the above examples are only used to illustrate the technical solution of the present disclosure, and not to limit it; although the present disclosure has been described in detail with reference to the aforementioned examples, those skilled in the art should understand that they can still modify the technical solutions recorded in the examples, or equivalently replace some or all of the technical features thereof; and these modifications or replacements do not make the essence of the corresponding technical solutions depart from the scope of the technical solutions of the various examples of the present disclosure, and they should all be covered within the scope of the claims and specifications of the present disclosure.

What is claimed is:

1. A Chinese medicine formula for treating tumors and/or pulmonary nodules, wherein it is prepared from the following raw materials:
Acanthopanax gracilistylus 20-30 g, Polygonum multiflorum 15-25 g, glossy ganoderma 15-25 g, snow pear 15-25 g, Gymnadenia conopsea 15-25 g, honey 450-550 g, Rheum officinale 15-25 g, Rhizoma polygonati 15-25 g, hanconggou 15-25 g and white wine 800-1200 ml.

2. The Chinese medicine formula according to claim 1, wherein it is prepared from the following raw materials:
Acanthopanax gracilistylus 25 g, Polygonum multiflorum 20 g, glossy ganoderma 20 g, snow pear 20 g, Gymnadenia conopsea 15-25 g, honey 500 g, Rheum officinale 20 g, Rhizoma polygonati 20 g, hanconggou 20 g and white wine 1000 ml.

3. The Chinese medicine formula according to claim 1, wherein the white wine is pure grain white wine and has an alcohol degree of 30°-53°.

4. The Chinese medicine formula according to claim 1, wherein the honey is mountain honey.

5. The Chinese medicine formula according to claim 1, wherein the tumors comprise lung cancer, liver cancer, kidney cancer, melanoma, gastric cancer, esophageal cancer, and lymphatic cancer.

6. A preparation method for the Chinese medicine formula according to claim 1, comprising the following steps:
(a) weighing each raw material;
(b) placing Acanthopanax gracilistylus, Polygonum multiflorum, glossy ganoderma, snow pear, Gymnadenia conopsea, honey, Rheum officinale, Rhizoma polygonati, and hanconggou into white wine, conducting soaking to obtain the Chinese medicinal formula.

7. The preparation method according to claim 6, wherein a temperature for soaking is 23-25° C. and a time for soaking is 30-40 days.

8. The preparation method according to claim 6, wherein the white wine is pure grain white wine and has an alcohol degree of 30°-53°.
